(19) United States
(12) Patent Application Publication
(10) Pub. No.: US 2010/0331282 A1
(43) Pub. Date: Dec. 30, 2010

(54) PYRIDINE DERIVATIVE CONTAINING ((PHOSPHONOOXY)METHYL)PYRIDINIUM RING, AND ANTIFUNGAL AGENT CONTAINING THESE DERIVATIVE

(76) Inventor: Masayuki MATSUKURA, Tsukuba-shi (JP)

Correspondence Address: BIRCH STEWART KOLASCH & BIRCH, PO BOX 747, FALLS CHURCH, VA, 22040-0747, US

(21) Appl. No.: 12/820,818

(22) Filed: Jun. 22, 2010

Related U.S. Application Data
(60) Provisional application No. 61/220,069, filed on Jun. 24, 2009.

(30) Foreign Application Priority Data
Jun. 24, 2009 (JP) 2009-149502

(51) Int. Cl. A61K 31/675; C07D 413/14; A61P 31/10

(52) U.S. Cl. 514/77; 546/22

(57) ABSTRACT

The present invention provides an antifungal agent that has excellent antifungal action, and is also superior in terms of its properties, and particularly its solubility in water and safety in an aqueous solution, and its in vivo pharmacokinetics and safety. According to the present invention, there is provided a compound represented by the following formula (I), or a salt thereof:

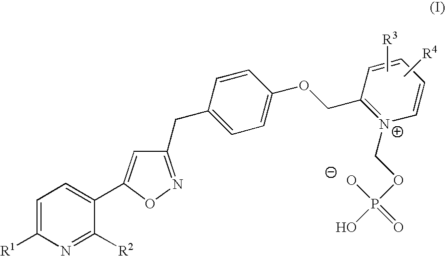

wherein R¹ represents a hydrogen atom, a halogen atom, an amino group, a C₁₋₆ alkyl group, a C₁₋₆ alkoxy group, or a C₁₋₆ alkoxy C₁₋₆ alkyl group;
R² represents a hydrogen atom, a C₁₋₆ alkyl group, an amino group, or a di-C₁₋₆ alkylamino group;
R³ represents a hydrogen atom, a halogen atom, or a C₁₋₆ alkyl group; and
R⁴ represents a hydrogen atom, a halogen atom, or a C₁₋₆ alkyl group.

### CROSS-REFERENCES TO RELATED APPLICATIONS

This application claims priorities of U.S. Provisional Application Ser. No. 61/220,067, filed Jun. 24, 2009, and Japanese Patent Application No. 2009-149502, filed Jun. 24, 2009, the disclosures of which are hereby incorporated by reference in entireties.

### TECHNICAL FIELD

The present invention relates to a novel pyridine derivative containing a ((phosphonooxy)methyl)pyridinium ring, and to an antifungal agents containing the derivative.

### BACKGROUND ART

In recent years, managements of opportunistic infections have become more and more significant more than ever because of an increase in the number of elderly people and immunocompromised patients as a result of advanced chemotherapies or the like. As demonstrated by the fact that opportunistic infections are occurring one after another by different avirulent pathogen, it is shown that the problem of infectious disease will not ends as long as there are underlying diseases that diminish the immune functions of patients. Consequently, new strategies for infectious diseases control, including the problem of drug-resistant pathogen, will be one of the important issues in the soon-to-come aged society.

In the field of antifungal agents, heretofore, for instance, amphotericine B which is based on a polyene skeleton, fluconazole, itraconazole and voriconazole which are based on an azole skeleton, or the like, have been developed for the treatment of deep seated mycoses. Most of pre-existing drugs already available commercially have similar mechanism of action, and currently, the appearance of azole-resistant fungi or the like has been problems.

In recent years, as a 1,3-β-glucan synthetase inhibitor with a novel mechanism, naturally occurring compound-derived cyclic hexapeptides caspofungin and micafungin or the like, have been developed; however, from the fact that these agents only exist in injectable form, they are not yet sufficient practically as antifungal agents.

Since there have been the situations that the pre-existing antifungal agents are insufficient for treatment of the deep seated mycoses, there is a demand and need for development of agents which are based on a novel mechanism and are of high safety. As the related art relevant to antifungal agents based on such a novel mechanism, Patent Documents 1 (WO 02/04626) and 2 (WO 05/033079) describe pyridine derivatives which demonstrates effects against the onset, progress, and persistence of infections by inhibiting the expression of cell wall proteins, inhibiting the cell wall assembly and also adhesion onto cells, and preventing pathogens from showing pathogenicity, with the process which transports GPI (Glycosylphosphatidylinositol)-anchored proteins to the cell wall being inhibited.

With this background, Patent Document 3 (WO 07/052,615) proposes a heterocycle-substituted pyridine derivative as an antifungal agent that has excellent antifungal action not found in conventional antifungal agents, and that is also superior in terms of physical properties, safety, and metabolic stability.

On the other hand, compounds and N-phosphoryloxymethyl prodrugs represented by the following formula have been disclosed as water-soluble prodrugs in Patent Documents 4 (U.S. Pat. No. 6,235,728 B1) and 5 (Japanese Patent Application Publication No. 2001-527083), respectively.

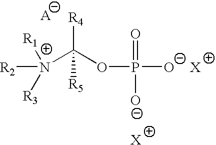

(wherein R¹, R², and R³ represent substituents containing ternary or secondary amines, each of R⁴ and R⁵ represents an organic or inorganic residue, and X represents a cationic organic or inorganic salt.)

Also, Patent Document 6 (WO 08/136,324) proposes a pyridine derivative substituted by a hetero ring and a phosphonoamino group, as a prodrug of an antifungal agent that is superior in terms of water solubility and safety.

### Patent Documents:

Patent document 1: WO 02/04626

Patent document 2: WO 05/033079

Patent document 3: WO 07/052,615

Patent document 4: U.S. Pat. No. 6,235,728 B1

Patent document 5: Japanese Patent Application Publication No. 2001-527083

Patent document 6: WO 08/136,324

### DISCLOSURE OF THE INVENTION

### Problems to be Solved by the Invention

However, to provide an even better method for treating fungal disease, we need to come up with an antifungal agent that is superior from the standpoints of solubility in water and stability in an aqueous solution, as well as safety.

In light of this situation, it is an object of the present invention to provide an antifungal agent that has excellent antifungal action, and is also superior in terms of physical properties, and particularly its solubility in water and stability in an aqueous solution, and its in vivo pharmacokinetics and safety.

### Means for Solving the Problems

As a result of diligent research into the above situation, the inventors perfected the present invention upon discovering that a pyridine derivative having a ((phosphonooxy)methyl)pyridinium ring represented by the following formula (I):

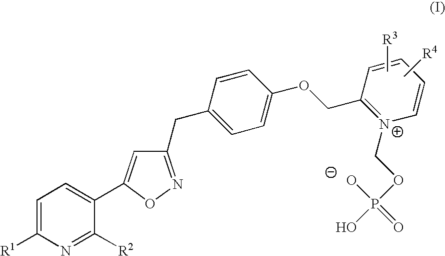

has an excellent antifungal action as a prodrug of a parent compound that is the active ingredient, and that is also superior in terms of its solubility in water and stability in an aqueous solution, and its in vivo pharmacokinetics and safety.

Specifically, the present invention provides:

[1] A compound represented by the following formula (I), or a salt thereof:

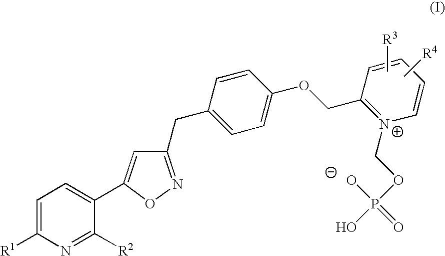

wherein R¹ represents a hydrogen atom, a halogen atom, an amino group, a C₁₋₆ alkyl group, a C₁₋₆ alkoxy group, or a C₁₋₆ alkoxy C₁₋₆ alkyl group;

R² represents a hydrogen atom, a C₁₋₆ alkyl group, an amino group, or a di-C₁₋₆ alkylamino group;

R³ represents a hydrogen atom, a halogen atom, or a C₁₋₆ alkyl group; and

R⁴ represents a hydrogen atom, a halogen atom, or a C₁₋₆ alkyl group.

[2] The compound or salt thereof according to item [1] above, wherein R² represents an amino group.
[3] The compound or salt thereof according to item [1] or [2] above, wherein R¹ represents a hydrogen atom.
[4] The compound or salt thereof according to item [1] or [2] above, wherein R¹ represents an amino group.
[5] The compound or salt thereof according to any one of items [1] to [4] above, wherein R³ represents a hydrogen atom, R⁴ represents a hydrogen atom, a halogen atom, or a C₁₋₆ alkyl group.
[6] A 2-((4-((5-(2-amino-3-pyridinyl)-3-isoxazolyl)methyl)phenoxy)methyl)-1-((phosphonooxy)methyl)pyridinium compound represented by the following formula, or a salt thereof:

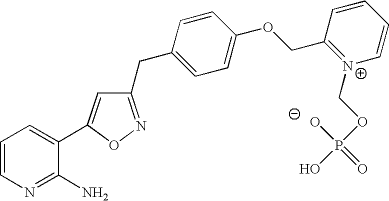

[7] A pharmaceutical composition comprising the compound according to any one of items [1] to [6] above, or a salt thereof.
[8] A medicament comprising the compound according to any one of items [1] to [6] above, or a salt thereof.
[9] An antifungal agent comprising, the compound according to any one of items [1] to [6] above, or a salt thereof, as an active ingredient.
[10] A method for preventing and/or treating a fungal infection comprising administering a pharmacologically effective amount of the compound according to any one of items [1] to [6], or a salt thereof.
[11] Use of the compound according to any one of items [1] to [6] above, or a salt thereof, for manufacturing an antifungal agent.

### ADVANTAGEOUS EFFECTS OF THE INVENTION

The compound represented by formula I (hereinafter sometimes referred to simply as “the compound according to the present invention”) is a prodrug of a parent compound that is an active ingredient, and 1) acts against the onset, development and persistence of infections by inhibiting fungal GPI biosynthesis, thereby inhibiting expression of cell wall proteins and blocking cell wall assembly while preventing the fungus from attaching to cells so that the pathogen cannot become pathogenic, and 2) is also superior in terms of physical properties, and particularly its solubility in water and stability in an aqueous solution, and its in vivo pharmacokinetics and safety, which makes this compound extremely useful in the prevention and treatment of fungal infections.

### DESCRIPTION OF THE DRAWINGS

FIG. 1 shows a graph of the change over time in the concentration of 2-((4-((5-(2-amino-3-pyridinyl)-3-isoxazolyl)methyl)phenoxy)methyl)-1-((phosphonooxy)methyl)pyridinium mono-trifluoroacetate obtained in Example 1 of the present invention and a parent compound (3-(3-(4-(pyridin-2-ylmethoxy)-benzyl)-isoxazol-5-yl)-pyridin-2-ylamine discussed in Reference Example 1), in a human liver S9 reaction solution;

FIG. 2 shows a graph of the change over time in the concentration of 2-((4-((5-(2-amino-3-pyridinyl)-3-isoxazolyl)methyl)phenoxy)methyl)-1-((phosphonooxy)methyl)pyridinium mono-trifluoroacetate obtained in Example 1 of the present invention and a parent compound (3-(3-(4-(pyridin-2-ylmethoxy)-benzyl)-isoxazol-5-yl)-pyridin-2-ylamine discussed in Reference Example 1), in a monkey liver S9 reaction solution; and

FIG. 3 shows a graph of the change over time in the concentration of 2-((4-((5-(2-amino-3-pyridinyl)-3-isoxazolyl)methyl)phenoxy)methyl)-1-((phosphonooxy)methyl)pyridinium mono-trifluoroacetate obtained in Example 1 of the present invention and a parent compound (3-(3-(4-(pyridin-2-ylmethoxy)-benzyl)-isoxazol-5-yl)-pyridin-2-ylamine discussed in Reference Example 1), in a reaction buffer solution.

### MODE FOR CARRYING OUT THE INVENTION

The present invention is explained below in more detail by reference to the symbols and the terms used herein being defined and the following examples. The present invention is not limited to or by the following embodiments, and various changes are possible within the scope of the invention.

Herein, a structural formula of a compound sometimes represents a certain isomer for convenience of description. However, compounds according to the present invention may include all possible isomers, such as structurally possible geometric isomers, optical isomers generated due to the presence of asymmetric carbons, stereoisomers, tautomers, and mixtures of isomers, and are not limited to formulae being used for the convenience of description, and may be either one of two isomers or a mixture of both isomers. Thus, the compounds according to the present invention may be either optically active compounds having an asymmetric carbon atom in their molecules or their racemates, and are not restricted to either of them but include both. Furthermore, the compounds according to the present invention may exhibit crystalline polymorphism, but likewise are not restricted to any one of these, but may be in any one of these crystal forms or exist as a mixture of two or more crystal forms. The compounds according to the present invention also include both anhydrous and solvates such as hydrated forms.

The term “C₁₋₆ alkyl group” used in the present specification refers to a straight-chain or branched-chain alkyl group with 1 to 6 carbon atoms which is a monovalent group induced by removal of any one hydrogen atom from an aliphatic hydrocarbon with 1 to 6 carbon atoms. Specifically, examples of “C₁₋₆ alkyl group” may includes a methyl group, an ethyl group, a n-propyl group, an isopropyl group, a n-butyl group, an isobutyl group, a sec-butyl group, a tert-butyl group, a n-pentyl group, an isopentyl group, a sec-pentyl group, a neopentyl group, a 1-methylbutyl group, a 2-methylbutyl group, a 1,1-dimethylpropyl group, a 1,2-dimethylpropyl group, a n-hexyl group, an isohexyl group, a 1-methylpentyl group, a 2-methylpentyl group, a 3-methylpentyl group, a 1,1-dimethylbutyl group, a 1,2-dimethylbutyl group, a 2,2-dimethylbutyl group, a 1,3-dimethylbutyl group, a 2,3-dimethylbutyl group, a 3,3-dimethylbutyl group, a 1-ethylbutyl group, a 2-ethylbutyl group, a 1,1,2,-trimethylpropyl group, a 1,2,2-trimethylpropyl group, a 1-ethyl-1-methylpropyl group, a 1-ethyl-2-methylpropyl group or the like, preferably a methyl group, an ethyl group, a n-propyl group, an isopropyl group, a n-butyl group, an isobutyl group, a sec-butyl group or a tert-butyl group or the like.

The term “C₁₋₆ alkoxy group” used in the present specification refers to a group in which an oxygen atom is bonded to terminus of the “C₁₋₆ alkyl group” defined above. Specifically, examples of “C₁₋₆ alkoxy group” may include a methoxy group, an ethoxy group, a n-propoxy group, an isopropoxy group, a n-butoxy group, an isobutoxy group, a sec-butoxy group, a tert-butoxy group, a n-pentyloxy group, an isopentyloxy group, a sec-pentyloxy group, a neopentyloxy group, a 1-methylbutoxy group, a 2-methylbutoxy group, a 1,1-dimethylpropoxy group, a 1,2-dimethylpropoxy group, a n-hexyloxy group, an isohexyloxy group, a 1-methylpentyloxy group, a 2-methylpentyloxy group, a 3-methylpentyloxy group, a 1,1-dimethylbutoxy group, a 1,2-dimethylbutoxy group, a 2,2-dimethylbutoxy group, a 1,3-dimethylbutoxy group, a 2,3-dimethylbutoxy group, a 3,3-dimethylbutoxy group, a 1-ethylbutoxy group, a 2-ethylbutoxy group, a 1,1,2-trimethylpropoxy group, a 1,2,2-trimethylpropoxy group, a 1-ethyl-1-methylpropxy group, a 1-ethyl-2-methylpropoxy group or the like, preferably a methoxy group, an ethoxy group, a n-propoxy group, an isopropoxy group, a n-butoxy group, an isobutoxy group, a sec-butoxy group, a tert-butoxy group or the like.

The term “C₁₋₆ alkoxy C₁₋₆ alkyl group” used in the present specification refers to a group in which any of the hydrogen atoms in a “C₁₋₆ alkyl group” as defined above has been replaced by a “C₁₋₆ alkoxy group” as defined above. Specifically, examples of “C₁₋₆ alkoxy C₁₋₆ alkyl group” may include a methoxymethyl group, an ethoxymethyl group, a n-propoxymethyl group, a methoxyethyl group, an ethoxyethyl group or the like.

The term “halogen atom” used in the present specification refers to a fluorine atom, a chlorine atom, a bromine atom or an iodine atom.

The term “di C₁₋₆ alkylamino group” used in the present specification refers to a group in which 2 hydrogen atoms of the amino group are replaced with the “C₁₋₆ alkyl groups” defined above being the same as or different from each other. Specifically, examples of the term “di C₁₋₆ alkylamino group” may include a N,N-dimethylamino group, a N,N-diethylamino group, a N,N-di-n-propylamino group, a N, N-di-isopropylamino group, a N,N-di-n-butylamino group, a N,N-isobutylamino group, a N,N-di-sec-butylamino group, a N,N-di-tert-butylamino group, a N-ethyl-N-methylamino group, a N-n-propylamino-N-methylamino group, a N-isopropyl-N-methylamino group, a N-n-butyl-N-methylamino group, a N-isobutyl-N-methylamino group, a N-sec-butyl-N-methylamino group, a N-tert-butyl-N-methlamino group or the like, preferably a N,N-dimethylamino group, a N,N-diethylamino group, N-ethyl-N-methylamino group or the like.

R¹ represents a hydrogen atom, a halogen atom, an amino group, a C₁₋₆ alkyl group, a C₁₋₆ alkoxy group, or a C₁₋₆ alkoxy-C₁₋₆ alkyl group, with a hydrogen atom or an amino group being particularly favorable.

R² represents a hydrogen atom, a C₁₋₆ alkyl group, an amino group, or a di-C₁₋₆ alkylamino group, with a hydrogen atom or an amino group being particularly favorable.

R³ represents a hydrogen atom, a halogen atom, or a C₁₋₆ alkyl group, with a hydrogen atom being particularly favorable.

R⁴ represents a hydrogen atom, a halogen atom, or a C₁₋₆ alkyl group, with a hydrogen atom being particularly favorable.

The term “salt” used in the present specification refers to a salt of an atom or a compound capable of forming a monovalent counter ion or a divalent counter ion. Examples thereof may include, but are not limited to, a salt of an inorganic acid (such as hydrochloric acid, hydrobromic acid, phosphoric acid, sulfuric acid, nitric acid, or the like), a salt of an organic acid (such as methanesulfonic acid, ethanesulfonic acid, benzenesulfonic acid, p-toluenesulfonic acid, fumaric acid, maleic acid, succinic acid, citric acid, malic acid, trifluoroacetic acid, or the like), a salt of an inorganic base (such as a sodium salt, potassium salt, calcium salt, lithium salt, or the like), and a salt of an organic base (such as a methylamine salt, ethylamine salt, t-butylamine salt, cyclohexylamine salt, N-methyl-D-glucamine salt, lysine salt, salt of piperidine or morpholine, or the like). Mono- and bis-salts are included in the term “salt.” Salts of the compound according to the present invention encompass anhydrides and hydrates of these salts and other such solvates of these salts.

The term “antifungal agent” used in the present specification refers to a preventive agent or a therapeutic agent for fungal infection.

The compounds according to the present invention can be formulated into tablets, powders, fine granules, granules, coated tablets, capsules, syrups, troches, inhalants, suppositories, injections, ointments, eye ointments, tapes, eye drops, nose drops, ear drops, cataplasms, lotions or the like, by the conventional methods.

Such formulation can be achieved by using typical diluents, binders, lubricants, colorants, flavorants, and, as necessary, stabilizers, emulsifiers, absorbefacients, surfactants, pH modulators, preservatives, antioxidants or the like, and materials commonly used as ingredients of pharmaceutical preparations according to the conventional methods. For example, an oral preparation can be produced by combining a compound of the present invention or a pharmaceutically acceptable salt thereof with a diluent, and if required, a binder, a disintegrating agent, a lubricant, a colorant, a flavorant or the like, and formulating the mixture into powders, fine granules, granules, tablets, coated tablets, capsules or the like according to the conventional methods.

Examples of the materials may include animal and vegetable oils such as soy bean oil, beef tallow, and synthetic glyceride; hydrocarbons such as liquid paraffin, squalane, and solid paraffin; ester oils such as octyldodecyl myristate and iso-propyl myristate; higher alcohols such as cetostearyl alcohol and behenyl alcohol; silicone resins; silicone oils; surfactants such as polyoxyethylene fatty acids ester, sorbitan fatty acid ester, polyoxyethylene sorbitan fatty acid ester, polyoxyethylene hydrogenated castor oil, and polyoxyethylene polyoxypropylene block co-polymer; water-soluble polymers such as hydroxyethyl cellulose, polyacrylic acid, carboxyvinyl polymer, polyethylene glycol, polyvinylpyrrolidone, and methytl cellulose; lower alcohols such as ethanol and isopropanol; polyhydric alcohols such as glycerol, propylene glycol, dipropylene glycol, and sorbitol; sugars such as glucose and sucrose; inorganic powder such as anhydrous silicic acid, magnesium aluminum silicate, and aluminum silicate; and pure water. Examples of the diluents may include lactose, corn starch, white sugar, glucose, mannitol, sorbitol, crystalline cellulose, silicon dioxide or the like. Examples of the binders may include polyvinyl alcohol, polyvinyl ether, methylcellulose, ethylcellulose, gum Arabic, tragacanth, gelatin, shellac, hydroxypropyl methylcellulose, hydroxypropyl cellulose, polyvinylpyrrolidone, polypropylene glycol-polyoxyethylene block co-polymer, and meglumine or the like. Examples of disintegrating agents may include starch, agar, gelatin powder, crystalline cellulose, calcium carbonate, sodium hydrogencarbonate, calcium citrate, dextrin, pectin, calcium carboxymethyl cellulose or the like. Examples of lubricants may include magnesium stearate, talc, polyethylene glycol, silica, hydrogenated vegetable oil or the like. Examples of colorants may include those pharmaceutically acceptable. Examples of flavorants may include cocoa powder, peppermint camphor, aromatic powder, peppermint oil, Borneo camphor, cinnamon powder or the like. Tablets and granules may be coated with sugar, or if required, other appropriate coatings can be made. Solutions, such as syrups or injectable preparations, to be administered can be formulated by combining a compound according to the present invention with a pH modulator, a solubilizing agent, an isotonizing agent or the like, and if required, with an auxiliary solubilizing agent, a stabilizer or the like, according to the conventional methods. Methods for manufacturing external preparations are not limited and such preparations can be manufactured by the conventional methods. Specifically, various materials typically used for manufacturing pharmaceuticals, quasi drugs, cosmetics or the like can be used as base materials for the external formulation. More specifically, examples of base materials to be used may include animal and vegetable oils, minerals oils, ester oils, wax, higher alcohols, fatty acids, silicone oil, surfactants, phospholipids, alcohols, polyhydric alcohols, water-soluble polymers, clay minerals, pure water or the like. Furthermore, external preparations of the present invention can contain, as required, pH modulators, antioxidants, chelating agents, antibacterial/antifungal agents, colorants, odoriferous substances or the like. But this does not limit the type of base materials that are to be used in the external preparations of the present invention. If required, the preparation may contain differentiation inducers, blood flow improving agents, antimicrobial agents, antiphologistics, cell activators, vitamins, amino acids, humectants, keratolytic agents or the like. The amount of the base materials listed above is adjusted within a concentration range used for producing typical external preparations.

When administering the compound of the present invention, the forms of the compounds are not limited in particular, and the compound can be given orally or parenterally by the conventional method. For instance, the compound can be administered as a dosage form such as tablets, powders, granules, capsules, syrups, troches, inhalants, suppositories, injections, ointments, eye ointments, tapes, eye drops, nasal drops, ear drops, cataplasms and lotions.

Dose of a medicament according to the present invention can be selected appropriately according to symptom severity, age, sex, body weight, forms of administration, type of salts, specific type of disease or the like.

The does varies remarkably depending on the patient's disease, symptom severity, age and sex, drug susceptibility or the like. An oral preparation according to the present invention can be generally administered once or several time at a dose of from 1 to 10000 mg/adult/day, preferably from 10 to 2000 mg/adult/day. An injection according to the present invention can be generally administered at a dose of from 0.1 to 10000 mg/adult/day, preferably from 1 to 2000 mg/adult/day.

### General Manufacturing Method

The method for manufacturing the compound represented by formula I (hereinafter referred to as compound I) will be described. In the manufacturing method discussed below, compounds I-1, I-2, and I-3 are described as typical examples of the compounds encompassed by compound I.

### General Manufacturing Method for Manufacturing Phosphoric Ester

### Manufacturing Method 1: Method for Manufacturing Compound I-1

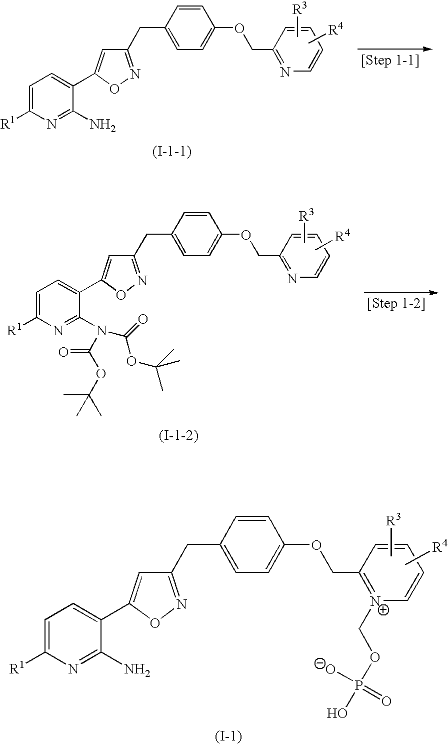

(wherein R¹, R³, and R⁴ are defined the same as above, excluding the compounds that R¹ represents an amino group.)

Compound I-1-1 can be manufactured using the method described in the reference examples given below. Compound I-1-1 can be also manufactured by the method described in for example, WO 2007/052615 A1.

### [Step 1-1]

This step is a step in which compound I-1-2 is obtained by reacting compound I-1-1 with di-tert-butyl dicarbamate in the presence of a base catalyst.

There are no particular restrictions on the solvent used in reacting compound I-1-1 with di-tert-butyl dicarbamate, so long as it can dissolve the starting raw materials to a certain extent and will not impede the reaction, but examples thereof may include methylene chloride, chloroform, and other such halogenated hydrocarbon-based solvents; tetrahydrofuran, diethyl ether, and other such ether-based solvents; ethyl acetate and other such ester-based solvents; acetonitrile, tetramethylene sulfolane, or mixtures of these solvents. The di-tert-butyl dicarbamate is used in an amount of from 2 to 20 equivalents based on compound I-1-1. For example, 4-dimethylaminopyridine is used as a base catalyst in an amount of from 0.001 to 0.3 equivalent. An organic base such as triethylamine or the like may also be added in an amount of from 1 to 2 equivalents. The reaction temperature is from 0° C. to 60° C., and preferably from 4° C. to room temperature. The reaction time is from 1 to 72 hours.

### [Step 1-2]

This step is a step in which compound I-1 is obtained by reacting compound I-1-2 with phosphoric acid di-tert-butyl ester chloromethyl ester in the presence of sodium iodide as an early stage, and then performing an acid treatment.

There are no particular restrictions on the solvent used in reacting compound I-1-2 with phosphoric acid di-tert-butyl ester chloromethyl ester in the presence of sodium iodide, so long as it can dissolve the starting raw materials to a certain extent and will not impede the reaction, but examples thereof may include methylene chloride, chloroform, and other such halogenated hydrocarbon-based solvents; tetrahydrofuran, diethyl ether, and other such ether-based solvents; ethyl acetate and other such ester-based solvents; acetonitrile, tetramethylene sulfolane, or mixtures of these solvents. The use of tetrahydrofuran or acetonitrile is preferable. The phosphoric acid di-tert-butyl ester chloromethyl ester is used in an amount of from 1 to 10 equivalents, and preferably from 1 to 2 equivalents, based on compound I-1-2. Sodium iodide can be used in an amount of from 1 to 10 equivalents, and preferably from 1 to 2 equivalents, based on compound I-1-2. The reaction temperature is from 0° C. to 60° C., and preferably from 4° C. to room temperature. The reaction time is from 1 to 72 hours.

The acid used for the acid treatment can be, for example, an organic acid such as trifluoroacetic acid, or a mineral acid such as hydrochloric acid, and the use of trifluoroacetic acid is preferred. In the acid treatment, the acid may be added directly to the reaction solvent at an early stage, or the solvent may first be evaporated under a reduced pressure, and then replaced the solvent with a suitable solvent such as dichloromethane followed by adding the acid. The reaction temperature is from −10° C. to room temperature, and the reaction time is from 5 minutes to 2 hours.

### Manufacturing Method 2: Method for Manufacturing Compound I-2

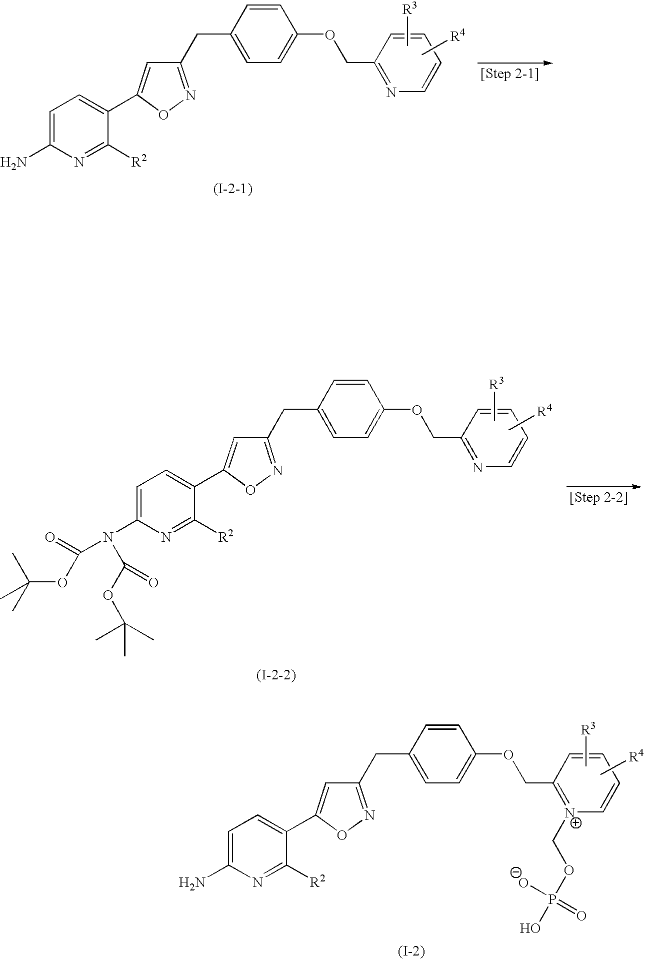

(wherein R², R³, and R⁴ are defined the same as above, excluding the compounds that R² represents an amino group.)

Compound I-2-1 can be manufactured by the method described in WO 2007/052615 A1.

### [Step 2-1]

This step is a step in which compound I-2-2 is obtained by reacting compound I-2-1 with di-tert-butyl dicarbamate in the presence of a base catalyst.

There are no particular restrictions on the solvent used in reacting compound I-2-1 with di-tert-butyl dicarbamate, so long as it can dissolve the starting raw materials to a certain extent and will not impede the reaction, but examples thereof may include methylene chloride, chloroform, and other such halogenated hydrocarbon-based solvents; tetrahydrofuran, diethyl ether, and other such ether-based solvents; ethyl acetate and other such ester-based solvents; acetonitrile, tetramethylene sulfolane, or mixtures of these solvents. The di-tert-butyl dicarbamate is used in an amount of from 2 to 20 equivalents based on compound I-2-1. For example, 4-dimethylaminopyridine is used as a base catalyst in an amount of from 0.001 to 0.3 equivalent. An organic base such as triethylamine or the like may also be added in an amount of from 1 to 2 equivalents. The reaction temperature is from 0° C. to 60° C., and preferably from 4° C. to room temperature. The reaction time is from 1 to 72 hours.

### [Step 2-2]

This step is a step in which compound I-2 is obtained by reacting compound I-2-2 with phosphoric acid di-tert-butyl ester chloromethyl ester in the presence of sodium iodide as an early stage, and then performing an acid treatment.

There are no particular restrictions on the solvent used in reacting compound I-2-2 with phosphoric acid di-tert-butyl ester chloromethyl ester in the presence of sodium iodide, so long as it can dissolve the starting raw materials to a certain extent and will not impede the reaction, but examples thereof may include methylene chloride, chloroform, and other such halogenated hydrocarbon-based solvents; tetrahydrofuran, diethyl ether, and other such ether-based solvents; ethyl acetate and other such ester-based solvents; acetonitrile, tetramethylene sulfolane, or mixtures of these solvents. The use of tetrahydrofuran or acetonitrile is preferable. The phosphoric acid di-tert-butyl ester chloromethyl ester can be used in an amount of from 1 to 10 equivalents, and preferably from 1 to 2 equivalents, based on compound I-2-2. Sodium iodide can be used in an amount of from 1 to 10 equivalents, and preferably from 1 to 2 equivalents, based on compound I-2-2. The reaction temperature is from 0° C. to 60° C., and preferably from 4° C. to room temperature. The reaction time is from 1 to 72 hours.

The acid used for the acid treatment can be, for example, an organic acid such as trifluoroacetic acid, or a mineral acid such as hydrochloric acid, and the use of trifluoroacetic acid is preferred. In the acid treatment, the acid may be added directly to the reaction solvent at an early stage, or the solvent may first be evaporated under a reduced pressure, and then replaced the solvent with a suitable solvent such as dichloromethane followed by adding the acid. The reaction temperature is from −10° C. to room temperature, and the reaction time is from 5 minutes to 2 hours.

### Manufacturing Method 3: Method for Manufacturing Compound I-3

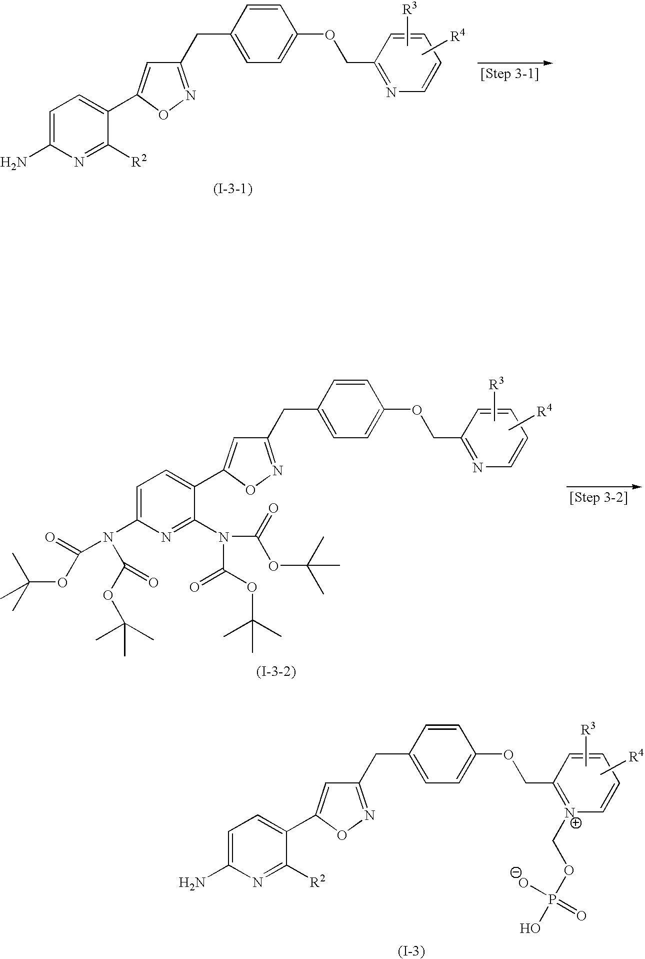

(wherein R³ and R⁴ are defined the same as above.)

Compound I-3-1 can be manufactured by the method described in for example, WO 2007/052615 A1.

### [Step 3-1]

This step is a step in which compound I-3-2 is obtained by reacting compound I-3-1 with di-tert-butyl dicarbamate in the presence of a base catalyst. In this step, compound I-3-2 can be obtained either by a single-stage reaction or by a multi-stage reaction in which a di-tert-butyl dicarbamate form of one amino group serves as an intermediate.

There are no particular restrictions on the solvent used in reacting compound I-3-1 with di-tert-butyl dicarbamate, so long as it can dissolve the starting raw materials to a certain extent and will not impede the reaction, but examples thereof may include methylene chloride, chloroform, and other such halogenated hydrocarbon-based solvents; tetrahydrofuran, diethyl ether, and other such ether-based solvents; ethyl acetate and other such ester-based solvents; acetonitrile, tetramethylene sulfolane, or mixtures of these solvents. The di-tert-butyl dicarbamate is used in an amount of from 2 to 20 equivalents based on compound I-3-1. 4-dimethylaminopyridine is used as a base catalyst in an amount of from 0.001 to 0.3 equivalent. An organic base such as triethylamine or the like may also be added in an amount of from 1 to 4 equivalents. The reaction temperature is from 0° C. to 60° C., and preferably from 4° C. to room temperature. The reaction time is from 1 to 72 hours.

### [Step 3-2]

This step is a step in which compound I-3 is obtained by reacting compound I-3-2 with phosphoric acid di-tert-butyl ester chloromethyl ester in the presence of sodium iodide as an early stage, and then performing an acid treatment.

There are no particular restrictions on the solvent used in reacting compound I-3-2 with phosphoric acid di-tert-butyl ester chloromethyl ester in the presence of sodium iodide, so long as it can dissolve the starting raw materials to a certain extent and will not impede the reaction, but examples thereof may include methylene chloride, chloroform, and other such halogenated hydrocarbon-based solvents; tetrahydrofuran, diethyl ether, and other such ether-based solvents; ethyl acetate and other such ester-based solvents; acetonitrile, tetramethylene sulfolane, or mixtures of these solvents. The use of tetrahydrofuran or acetonitrile is preferable. The phosphoric acid di-tert-butyl ester chloromethyl ester can be used in an amount of from 1 to 10 equivalents, and preferably from 1 to 2 equivalents, based on compound I-3-2. Sodium iodide can be used in an amount of from 1 to 10 equivalents, and preferably from 1 to 2 equivalents, based on compound I-3-2. The reaction temperature is from 0° C. to 60° C., and preferably from 4° C. to room temperature. The reaction time is from 1 to 72 hours.

The acid used for the acid treatment can be, for example, an organic acid such as trifluoroacetic acid, or a mineral acid such as hydrochloric acid, and the use of trifluoroacetic acid is preferred. In the acid treatment, the acid may be added directly to the reaction solvent at an early stage, or the solvent may first be evaporated under a reduced pressure, and then replaced the solvent with a suitable solvent such as dichloromethane followed by adding the acid. The reaction temperature is from −10° C. to room temperature, and the reaction time is from 5 minutes to 2 hours.

### EXAMPLES

The compound according to the present invention can be manufactured by the methods described in the following examples, reference examples, and manufacturing examples, for example. These are only given for illustrative purposes, however, and the compound according to the present invention is in no way limited to or by the following specific examples.

The abbreviation used in the description of the examples, reference examples, manufacturing examples, and so forth is defined as follows.

TFA: trifluoroacetic acid

### Reference Example 1

### 3-(3-(4-(Pyridin-2-ylmethoxy)-benzyl)-isoxazol-5-yl)-pyridin-2-ylamine

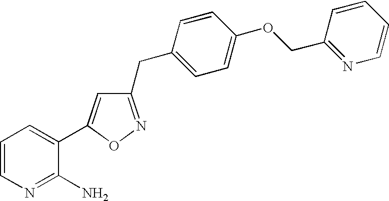

To a mixture of 4-(5-(2-amino-pyridin-3-yl)-isoxazol-3-ylmethyl)-phenol (4.2 mg, 0.016 mmol) described in Manufacturing Example 1-1-5 and methanol (0.4 mL) was added a 1N sodium hydroxide aqueous solution (16 μL, 0.016 mmol) was added, which was concentrated under a reduced pressure. To a mixture of the residue and N,N-dimethylformamide (0.5 mL) was added 3.1 mg (0.019 mmol) of 2-picolyl chloride (3.1 mg, 0.019 mmol), which was stirred for two hours at room temperature. The reaction mixture was directly purified by reverse phase high performance liquid chromatography (using an acetonitrile-water-based mobile phase (containing 0.1% trifluoroacetic acid)), which gave the titled compound (3.6 mg, 39%) as a di-trifluoroacetate.

MS m/e (ESI) 359.16 (MH⁺)

As another method, the compound of Reference Example 1 was obtained as follows.

To a mixture of 4-(5-(2-amino-pyridin-3-yl)-isoxazol-3-ylmethyl)-phenol (2.97 g, 11.1 mmol) described in Manufacturing Example 1-1-5, tetrahydrofuran (100 mL), and acetone (100 mL) was added a 5N sodium hydroxide aqueous solution (2.22 mL, 11.1 mmol). The reaction mixture was subjected to the ultrasonic treatment for 30 seconds, which was concentrated under a reduced pressure. To a mixture of the residue and N,N-dimethylformamide (50 mL) was added 2-picolyl chloride (3.64 g, 22.2 mmol), which was stirred for 2.5 hours at 60° C. The reaction mixture was returned to room temperature and quenched with water, and the reaction mixture was then extracted with ethyl acetate. The organic layer was washed with water and saturated brine, and dried over anhydrous magnesium sulfate. The solvent was evaporated under a reduced pressure, and the residue was purified by NH-silica gel column chromatography (heptane:ethyl acetate=1:1), which gave the titled compound (2.73 g, 67%).

¹H-NMR spectrum (CDCl₃) δ (ppm): 4.00 (2H, s), 5.20 (2H, s), 5.37 (2H, brs), 6.24 (1H, s), 6.71 (1H, dd, J=4.8, 7.6 Hz), 6.95-6.97 (2H, m), 7.20-7.22 (2H, m), 7.52 (d, 1H, d, J=1.9 Hz), 7.69-7.74 (3H, m), 8.13-8.15 (1H, m), 8.60 (1H, d, J=4.4 Hz).

The starting material, 4-(5-(2-amino-pyridin-3-yl)-isoxazol-3-ylmethyl)-phenol, was synthesized by the following method.

### Manufacturing Example 1-1-1

### 1-Benzyloxy-4-((E)-2-nitro-vinyl)-benzene

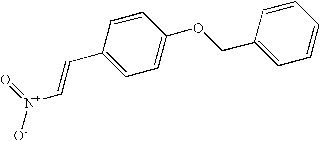

To a mixture of 4-benzyloxybenzaldehyde (1.0 g, 4.7 mmol), sodium methoxide (150 μL, 0.74 mmol in a 28% methanol solution), and methanol (10 mL) were added nitromethane (330 μL, 6.1 mmol) and sodium methoxide (1.0 mL (4.9 mmol in a 28% methanol solution)) at 0° C., which was stirred for 10 minutes at room temperature. The reaction mixture was cooled to 0° C., and a 5 N hydrochloric acid aqueous solution (20 mL) was added thereto at the same temperature. The reaction mixture was stirred for 15 minutes at room temperature. The precipitated solids were collected by filtering, which gave the titled compound (1.2 g, 100%).

¹H-NMR spectrum (DMSO-d₆) δ (ppm): 5.20 (2H, s), 7.10-7.14 (2H, m), 7.32-7.48 (5H, m), 7.82-7.85 (2H, m), 8.12 (2H, dd, J=13.5, 18.2 Hz).

### Manufacturing Example 1-1-2

### 1-Benzyloxy-4-(2-nitro-ethyl)-benzene

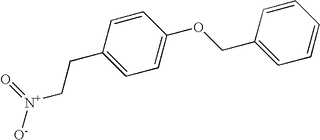

To a mixture of 1-benzyloxy-4-((E)-2-nitro-vinyl)-benzene (1.0 g, 3.9 mmol) described in Manufacturing Example 1-1-1, acetic acid (1 mL), and dimethyl sulfoxide (17 mL) was added sodium borohydride (250 mg, 6.3 mmol) while suitable cooling, which was stirred for 40 minutes at room temperature. This reaction mixture was added with water and partitioned into ethyl acetate and water. The organic layer was washed with water and saturated brine, and dried over anhydrous magnesium sulfate, and the solvent was evaporated under a reduced pressure. The residue was purified by NH-silica gel column chromatography (ethyl acetate:heptane=1:3), which gave the titled compound (710 mg, 70%).

¹H-NMR spectrum (CDCl₃) δ (ppm): 3.26 (2H, t, J=7.2 Hz), 4.56 (2H, t, J=7.2 Hz), 5.04 (2H, s), 6.92 (2H, d, J=8.4 Hz), 7.11 (2H, d, J=8.8 Hz), 7.30-7.42 (5H, m).

### Manufacturing Example 1-1-3

### 4-Benzyloxy-phenyl-acetohydroxymoyl chloride

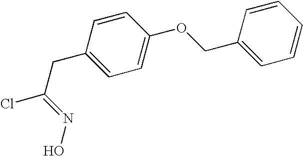

To a mixture of 1-benzyloxy-4-(2-nitro-ethyl)-benzene (340 mg, 1.3 mmol) described in Manufacturing Example 1-1-2 and methanol (5 mL) was added lithium methoxide (100 mg, 2.6 mmol) at room temperature, which was stirred for 15 minutes at room temperature. The reaction mixture was concentrated under a reduced pressure, and methylene chloride (4 mL) and tetrahydrofuran (2 mL) were added to the residue. Titanium(IV) chloride was added at −78° C. to the reaction mixture followed by stirring for 50 minutes at 0° C. The reaction mixture was cooled to −78° C., water (5 mL) was then added, and the temperature was gradually raised to room temperature. The reaction mixture was partitioned into ethyl acetate and water. The organic layer was washed with saturated brine, and the solvent was evaporated under a reduced pressure. The residue was purified by neutral silica gel column chromatography (ethyl acetate:heptane=1:3), which gave the titled compound (310 mg, 84%).

¹H-NMR spectrum (CDCl₃) δ (ppm): 3.83 (2H, s), 5.07 (2H, s), 6.94-6.98 (2H, m), 7.17-7.21 (2H, m), 7.32-7.44 (5H, m).

### Manufacturing Example 1-1-4

### 3-(3-(4-Benzyloxy-benzyl)-isoxazol-5-yl)-pyridin-2-ylamine

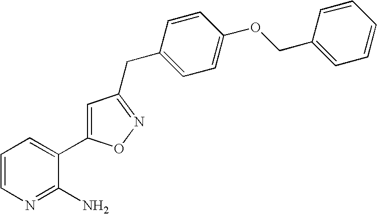

To a mixture of 4-benzyloxy-phenyl-acetohydroxymoyl chloride (1.2 g, 4.4 mmol) described in Manufacturing Example 1-1-3 and tetrahydrofuran (34 mL) were added 3-ethynyl-pyridin-2-ylamine (260 mg, 2.2 mmol) described in Manufacturing Example 1-2-5 and triethylamine (3.0 mL, 22 mmol) at 0° C., which was stirred for 1 hour at room temperature. Water was added at room temperature to the reaction mixture, and extraction was performed with ethyl acetate and tetrahydrofuran (2:1). The organic layer was washed with saturated brine, and the solvent was evaporated under a reduced pressure. The residue was purified by NH-silica gel column chromatography (ethyl acetate:heptane=1:3), which gave the titled compound (240 mg, 15%).

¹H-NMR spectrum (CDCl₃) δ (ppm): 4.00 (2H, s), 5.05 (2H, s), 5.41 (2H, s), 6.24 (1H, s), 6.71 (1H, dd, J=4.9, 7.6 Hz), 6.93-6.97 (2H, m), 7.18-7.22 (2H, m), 7.31-7.44 (5H, m), 7.70 (1H, dd, J=1.7, 7.6 Hz), 8.13 (1H, dd, J=1.8, 4.9 Hz).

### Manufacturing Example 1-1-5

### 4-(5-(2-Amino-pyridin-3-yl)-isoxazol-3-ylmethyl)-phenol

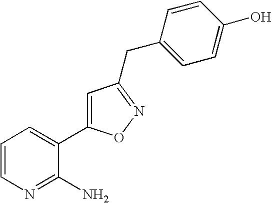

To a mixture of 3-(3-(4-benzyloxy-benzyl)-isoxazol-5-yl)-pyridin-2-ylamine (32 mg, 0.090 mmol) described in Manufacturing Example 1-1-4 and trifluoroacetic acid (1 mL) was added thioanisole (45 mg, 0.36 mmol) at room temperature, which was stirred for 2 hours at the same temperature. The reaction mixture was added to a mixture of saturated sodium hydrogencarbonate and ethyl acetate. The organic layer was separated and washed with saturated brine, and the solvent was devaporated under a reduced pressure. The residue was purified by silica gel column chromatography (ethyl acetate:heptane=4:1), which gave the titled compound (24 mg, 100%).

¹H-NMR spectrum (DMSO-d₆) δ (ppm): 3.90 (2H, s), 6.25 (2H, brs), 6.68-6.72 (3H, m), 6.76 (1H, s), 7.11 (2H, d, J=8.6 Hz), 7.87 (1H, dd, J=1.5, 7.7 Hz), 8.10 (1H, brs), 9.29 (1H, s).

The starting material, 3-ethynyl-pyridin-2-ylamine, was synthesized by the following method.

### Manufacturing Example 1-2-1

### 2,2-Dimethyl-N-pyridin-2-yl-propionamide

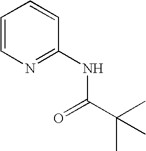

To a methylene chloride solution (500 mL) of 2-aminopyridine (50.0 g, 531 mmol) were added triethylamine (81.4 mL, 584 mmol) and pivaloyl chloride (71.9 mL, 584 mmol) at 0° C., which was stirred for 4 hours and 30 minutes at room temperature. The reaction solution was partitioned into water and methylene chloride. The organic layer was washed with water and saturated brine and dried over anhydrous magnesium sulfate, and the solvent was evaporated under a reduced pressure. Potassium carbonate (73.4 g, 531 mmol) was added to 300 mL of thus obtained residue methanol solution at 0° C., which was stirred at room temperature for 90 minutes. This reaction solution was partitioned into water and ethyl acetate. The organic layer was washed with saturated brine, and dried over anhydrous magnesium sulfate, the solvent was evaporated under a reduced pressure. Heptane (300 mL) was added to the residue, the precipitated solids were collected by filtering, which gave the titled compound (80.2 g, 85%). The filtrate was then concentrated under a reduced pressure, and the residue was purified by silica gel column chromatography (heptane:ethyl acetate=2:1), which gave the titled compound (12.2 g, 13%).

¹H-NMR spectrum (DMSO-d₆) δ (ppm): 1.22 (9H, s), 7.06-7.09 (1H, m), 7.72-7.77 (1H, m), 8.01-8.03 (1H, m), 8.29-8.31 (1H, m), 9.71 (1H, s).

### Manufacturing Example 1-2-2

### N-(3-Iodo-pyridin-2-yl)-2,2-dimethyl-propionamide

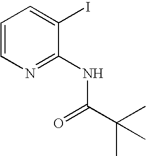

To a mixture of 2,2-dimethyl-N-pyridin-2-yl-propionamide (3.0 g, 17 mmol) described in Manufacturing Example 1-2-1, N,N,N′,N′-tetramethylethylenediamine (6.3 mL, 42 mmol), and tetrahydrofuran (60 mL) was added n-butyl lithium (30 mL, 47 mmol; in a 1.6 M n-hexane solution) dropwise at −78° C., which was stirred overnight at 0° C. Iodine (6.8 g, 27 mmol) was added at −78° C. to the reaction mixture, which was stirred for 1.5 hours at 0° C. Water and a saturated sodium thiosulfate aqueous solution were added to the reaction mixture, and extraction was performed with ethyl acetate. The organic layer was washed with saturated brine, and the solvent was evaporated under a reduced pressure. The residue was purified by silica gel column chromatography (ethyl acetate:heptane=2:1), which gave the titled compound (2.9 g, 57%).

¹H-NMR spectrum (CDCl₃) δ (ppm): 1.38 (9H, s), 6.85 (1H, dd, J=4.8, 7.9 Hz), 7.94 (1H, brs), 8.11 (1H, dd, J=1.7, 7.9 Hz), 8.46 (1H, dd, J=1.7, 4.6 Hz).

### Manufacturing Example 1-2-3

### 3-Iodo-pyridin-2-ylamine

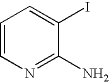

A mixture of N-(3-iodo-pyridin-2-yl)-2,2-dimethyl-propionamide (66.2 g, 218 mmol) described in Manufacturing Example 1-2-2, a 5 N sodium hydroxide aqueous solution (200 mL), and methanol (200 mL) was heated to reflux, which was stirred for 1 hour and 20 minutes. The reaction solution was returned to room temperature and partitioned into water and ethyl acetate. The aqueous layer was extracted three times with ethyl acetate. This was combined with the organic layer, washed with saturated brine, and dried over anhydrous sodium sulfate. The sodium sulfate was filtered off, and the solvent was evaporated under a reduced pressure, which gave the titled compound (41.2 g, 85.9%).

¹H-NMR spectrum (DMSO-d₆) δ (ppm): 6.00 (2H, brs), 6.32 (1H, dd, J=4.8 Hz, 7.2 Hz), 7.87 (1H, d, J=7.2 Hz), 7.92 (1H, d, J=4.8 Hz).

### Manufacturing Example 1-2-4

### 3-Trimethylsilanylethynyl-pyridin-2-ylamine

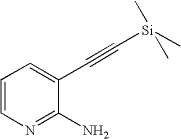

To a mixture of 3-iodo-pyridin-2-ylamine (40.2 g, 183 mmol) described in Manufacturing Example 1-2-3, trimethylsilylacetylene (51.7 mL, 366 mmol), copper(I) iodide (3.49 g, 18.3 mmol), N,N-diisopropylethylamine (63.7 mL, 366 mmol), and N-methylpyrrolidone (200 mL) was added tetrakis(triphenylphosphine) palladium(0) (10.6 g, 9.15 mmol), which was stirred for 3 hours and 10 minutes at room temperature under a nitrogen gas flow. Water was added to the reaction solution, which was then extracted four times with ethyl acetate. The solvent was evaporated under a reduced pressure, and the residue was purified by NH-silica gel chromatography (heptane:ethyl acetate=4:1). The solution thus obtained was concentrated under a reduced pressure, and the residue was purified by silica gel chromatography (heptane:ethyl acetate=2:1, then 1:1), which gave the titled compound (28.1 g, 80.7%).

¹H-NMR spectrum (DMSO-d₆) δ (ppm): 0.25 (9H, s), 6.09 (2H, brs), 6.51-6.57 (1H, m), 7.50-7.55 (1H, m), 7.95-7.99 (1H, m).

### Manufacturing Example 1-2-5

### 3-Ethynyl-pyridin-2-ylamine

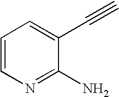

To a tetrahydrofuran solution (300 mL) of 3-trimethylsilanylethynyl-pyridin-2-ylamine (28.1 g, 148 mmol) described in Manufacturing Example 1-2-4 was added tetrabutylammonium fluoride (20 mL (20 mmol in a 1 M tetrahydrofuran solution)), which was stirred for 15 minutes at room temperature. Water was added to the reaction solution, which was then extracted four times with ethyl acetate. The organic layer was dried over anhydrous sodium sulfate, and the solvent was evaporated under a reduced pressure. The residue was purified by silica gel chromatography (heptane:ethyl acetate=1:1, then 1:2), which gave the titled compound (16.4 g, 93.7%).

¹H-NMR spectrum (DMSO-d₆) δ (ppm): 4.43 (1H, s), 6.14 (2H, brs), 6.53 (1H, dd, J=4.8 Hz, 7.2 Hz), 7.53 (1H, d, J=7.2 Hz), 7.96 (1H, d, J=4.8 Hz).

### Manufacturing Example 1-3-1

### 3-Trimethylsilanylethynyl-pyridin-2-ylamine

### Alternative Method to Manufacturing Example 1-2-4

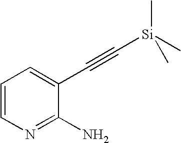

To a N-methylpyrrolidinone solution (120 mL) of 2-amino-3-bromopyridine (5.72 g, 33.1 mmol) were added triemthylsilyl acetylene (9.36 mL, 66.2 mmol), tetrakis(triphenylphosphine) palladium(0) (1.91 g, 1.66 mmol), copper(I) iodide (630 mg, 3.31 mmol), and N,N-diisopropylethylamine (11.5 mL, 66.2 mmol), which was stirred for 6 hours at 70° C. under a nitrogen atmosphere. Water was added to the reaction solution, and extraction was performed with ethyl acetate. The organic layer was washed with water and saturated brine and dried over anhydrous magnesium sulfate, and the solvent was evaporated under a reduced pressure. The residue was purified by silica gel column chromatography (heptane:ethyl acetate=2:1), which gave the titled compound (5.94 g, 94%).

¹H-NMR spectrum (DMSO-d₆) δ (ppm): 0.23 (9H, s), 6.07 (2H, brs), 6.51 (1H, dd, J=4.9, 7.5 Hz), 7.49 (1H, dd, J=1.8, 7.5 Hz), 7.94 (1H, dd, J=1.8, 4.9 Hz).

### Example 1

### 2-((4-((5-(2-Amino-3-pyridinyl)-3-isoxazolyl)methyl)phenoxy)methyl)-1-((phosphonooxy)methyl)pyridinium mono-trifluoroacetate

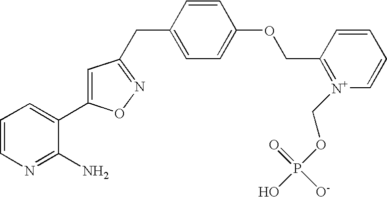

A mixture of di-tert-butyl-(3-(3-(4-(pyridin-2-ylmethoxy)benzyl)isoxazol-5-yl)pyridin-2-yl)imide dicarbonate (334 mg, 0.60 mmol)) described in Manufacturing Example 1-4-1, phosphoric acid di-tert-butyl ester chloromethyl ester (309 mg, 1.2 mmol), sodium iodide (134 mg, 0.90 mmol), and tetrahydrofuran (0.6 mL) were stirred for 3.5 hours at room temperature, trifluoroacetic acid (2 mL) was then added to this reaction solution, which was stirred for another 40 minutes at room temperature. The reaction solution was concentrated under a reduced pressure, aqueous sodium bicarbonate and ethyl acetate were added to the residue, and which was separated. Ethyl acetate was added to the aqueous layer, and which was separated again. The aqueous sodium bicarbonate thus obtained was gel filtered (CHP20P (made by Mitsubishi Kasei), water, then methanol elution), and then the eluate was concentrated until the amount of liquid was about 10 mL. The solution thus obtained was purified in an ODS column (H₂O:MeOH:TFA=500:50:0.5, then 500:200:0.7). The solvent was evaporated, and the residue was dissolved in a small amount of acetone, after which ethyl acetate was added thereto, which was concentrated, which gave the titled compound (53.93 mg) as a powdered solid.

¹H-NMR spectrum (DMSO-d₆) δ (ppm): 4.02 (2H, s), 5.73 (2H, s), 6.33 (2H, d, J=13.5 Hz), 6.84 (1H, dd, J=5.2, 7.6 Hz), 6.89 (1H, s), 7.13 (2H, d, J=8.8 Hz), 7.33 (2H, d, J=8.8 Hz), 8.07 (1H, dd, J=1.6, 7.6 Hz), 8.12 (1H, dd, J=1.6, 5.2 Hz), 8.15-8.22 (1H, m), 8.28 (1H, d, J=7.6 Hz), 8.73 (1H, ddd, J=1.6, 7.6, 7.6 Hz), 9.22 (1H, d=4.8 Hz).

The starting material, di-tert-butyl-(3-(3-(4-(pyridin-2-ylmethoxy)benzyl)isoxazol-5-yl)pyridin-2-yl)imide dicarbonate, was synthesized by the following method.

### Manufacturing Example 1-4-1

### Di-tert-butyl-(3-(3-(4-(pyridin-2-ylmethoxy)benzyl)isoxazol-5-yl)pyridin-2-yl)imide dicarbonate

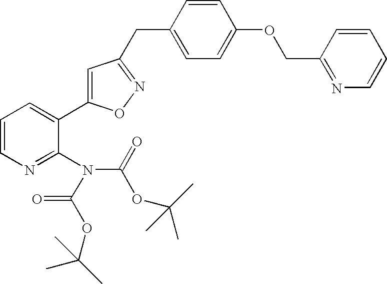

A mixture of 3-(3-(4-(pyridin-2-ylmethoxy)-benzyl)-isoxazol-5-yl)-pyridin-2-ylamine (300 mg, 0.84 mmol) described in Reference Example 1, di-tert-butyl dicarbonate (913 mg, 4.2 mmol), 4-dimethylaminopyridine (10 mg, 0.084 mmol), triethylamine (102 mg, 1.0 mmol), and tetrahydrofuran (7 mL) were stirred for 13.5 hours at room temperature. The reaction solution was purified by NH-silica gel column chromatography (heptane:ethyl acetate=1:1, then 1:2), which gave the titled compound (334 mg).

¹H-NMR spectrum (CDCl₃) δ (ppm): 1.24 (18H, s), 3.99 (2H, s), 5.18 (2H, s), 6.32 (1H, s), 6.90-6.95 (2H, m), 7.16-7.24 (3H, m), 7.41 (1H, dd, 4.8, 8.0 Hz), 7.51 (1H, d, 8.0 Hz), 7.71 (1H, ddd, J=2.0, 8.0, 8.0 Hz), 8.27 (1H, dd, J=2.0, 8.0 Hz), 8.56-8.61 (2H, m).

The compound according to the present invention represented by formula I is rapidly converted into a parent compound that is an active form having excellent antifungal activity, and is also superior in terms of its properties, and particularly its solubility in water and stability in an aqueous solution, and its safety, and is thus extremely useful as an agent for preventing or treating fungal infections.

### 1. Comparative Test of Solubility in Water

3-(3-(4-(Pyridin-2-ylmethoxy)-benzyl)-isoxazol-5-yl)-pyridin-2-ylamine described in Reference Example 1, which is the parent compound, and the compound of Example 1 were compared for solubility in a Britton-Robinson buffer (ion strength of 0.3) at 25° C. These results are given in Table 1.

TABLE 1

| | Solubility (mg/mL) | | |
|---|---|---|---|
| Test compound | pH 3 | pH 7 | pH 9 |
| Reference Example 1 (parent | 0.4 | <0.1 | <0.1 |
| compound) | | | |
| Compound of Example 1 | >70 | >70 | >70 |

As can be clear from the results given in Table 1, the compound of Example 1 was found to have markedly higher solubility in water than its parent compound in each of the pH regions.

2. Conversion into Parent Compound (Active Form) in Liver S9 Fraction

### (1) Preparation of Various Liver S9 Reaction Solutions

A suspension (pH of 7.4) containing human and monkey liver S9 fractions (with a protein concentration of 0.22 mg/mL), 0.5 mmol/L magnesium chloride, and 100 mmol/L tris-HCl was prepared over ice (various reaction solutions A). 30 μL of a 100 μmol/mL aqueous solution of the compound according to the present invention (the compound of Example 1) was added to the various reaction solutions A (for a final compound concentration of 10 μmol/L) to obtain various liver S9 reaction solutions (with a final protein concentration of 0.2 mg/mL), and these were stored on ice until (2) was carried out. A control sample was prepared (reaction buffer solution) by adding 30 μL of a 100 μmol/mL aqueous solution of the compound according to the present invention (the compound of Example 1) to 0.27 mL of a buffer (pH of 7.4) containing 0.5 mmol/L magnesium chloride and 100 mmol/L tris-HCl.

(2) Conversion into Parent Compound (Compound of Reference Example 1) in Various Liver S9 Reaction Solutions and a Reaction Buffer Solution

The various liver S9 reaction solutions and the reaction buffer solution of (1) were incubated at 37° C., samples were collected in 50 μL amounts each time at 0, 30, and 60 minutes, 100 μL of a methanol solution was added, and the reaction was halted.

(3) The Concentrations of the Compound According to the Present Invention (the Compound of Example 1) and the Parent Compound (the Compound of Reference Example 1) in the Reaction Solution were Quantified by LC-MS.

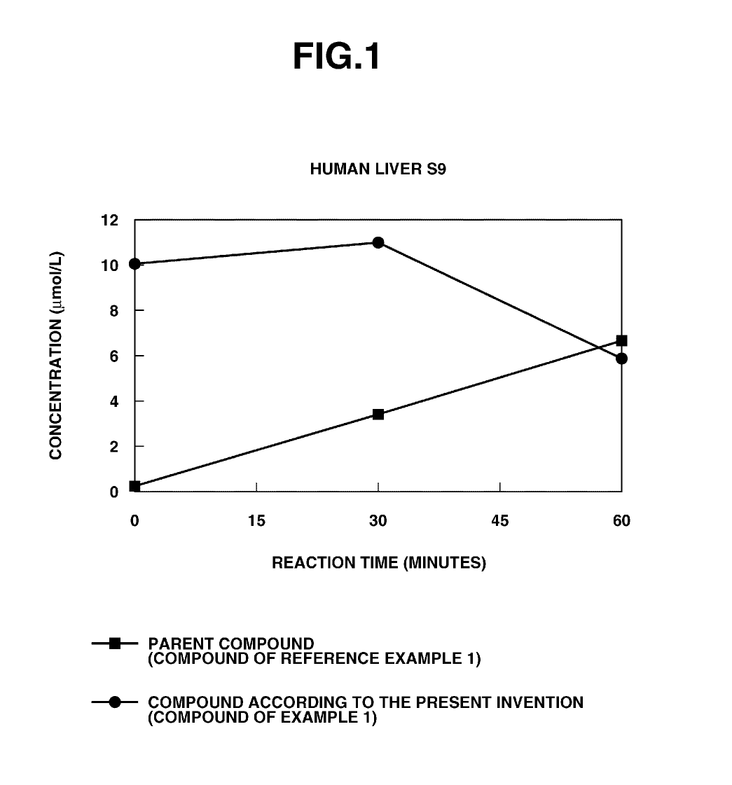

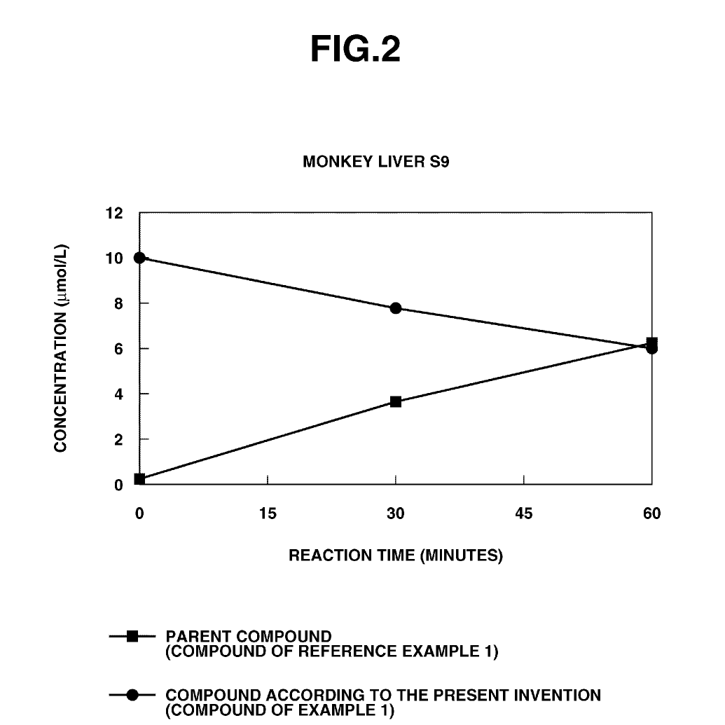

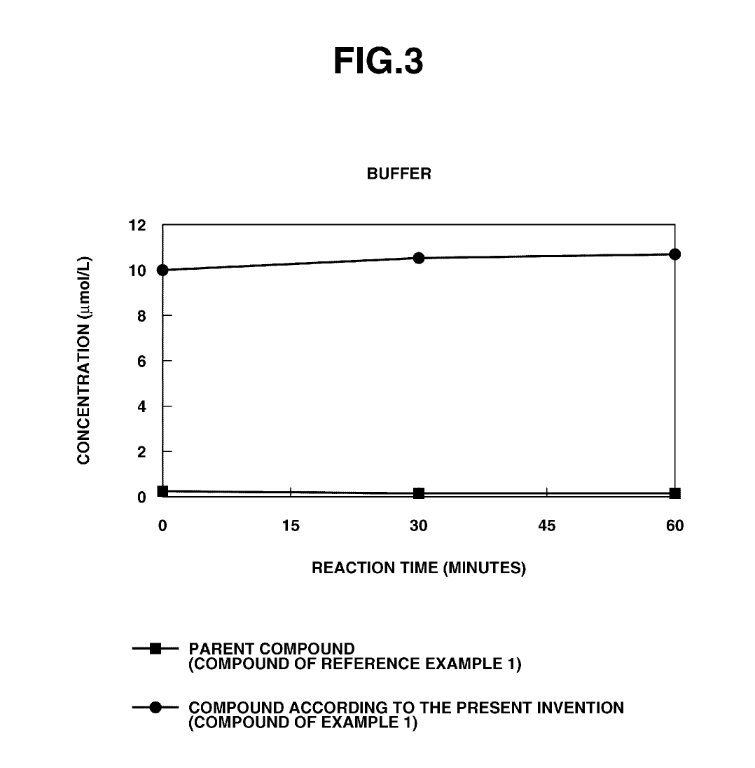

The concentrations of the compound according to the present invention (the compound of Example 1) and the parent compound (the compound of Reference Example 1) in the reaction solution were measured by the method described in 2. above. FIGS. 1 to 3 show graphs of the change over time in the concentrations of the compound of Example 1 and the compound of Reference Example 1 in various liver S9 reaction solutions and the reaction buffer solution. It can be seen from the results in FIGS. 1 to 3 that the compound according to the present invention (the compound of Example 1) was converted into the parent compound (the compound of Reference Example 1) in human and monkey liver S9 fractions. It was also confirmed that no conversion from the compound according to the present invention (the compound of Example 1) into the parent compound (the compound of Reference Example 1) was observed in the reaction buffer solution that did not contain a liver S9 fraction.

### 3. Anti-Candida Activity and Anti-Aspergillus Activity

### (1) Preparation of Fungal Suspension

For the C. albicans CAF2-1 strain, a fungal suspension from a standing culture for 48 hours at 30° C. in a Sabouraud dextrose liquid culture medium (SDB) was diluted with RPMI1640 medium to adjust a fungal suspension of 1.2×10³ cells/mL. For the A. fumigatus Tsukuba strain, −80° C. stored strain was diluted with RPMI1640 medium to adjust to a fungal suspension of 4.5×10³ cells/mL.

### (2) Preparation of an Agent Dilution Plate

Using a U-bottomed 96 well plate, 8 samples/plate (A to H) of sample dilution solutions were prepared. On the 2^{nd} to 12^{th} rows were dispensed 10 μl of dimethyl sulfoxide solution. Weighted sample was dissolved in dimethyl sulfoxide to prepare a 2.5 mg/mL solution, 20 μl of this solution was added to the first row of the prepared plate, and 12 steps of two-folded step dilutions (10 μl of solution+10 μl of dimethyl sulfoxide solution) were performed on the plate. This sample dilution solution was dispensed in the amount of 1 μl to a flat-bottomed 96 well plate for MIC measurement to prepare a sample dilution plate.

### (3) Inoculation of Fungal Suspension and Culture

The fungal suspension prepared in (1) was used in the amount of 99 mL/well to inoculate the flat-bottomed 96 well plate containing 1 μL/well of the test compound dilution prepared in (2), and a standing culture was carried out aerobically for 42-48 hours at 35° C.

### (4) MIC Measurement

The minimum concentration that clearly inhibited fungal growth as compared to the control by visual inspection was determined as the minimum inhibitory concentration (MIC).

The parent compound (the compound of Reference Example 1) were measured for anti-Candida activity and anti-Aspergillus activity by the measurement method in 3 above. These results are given in Table 2. It was confirmed from the results in Table 2 that the parent compound (the compound in Reference Example 1) had anti-Candida and anti-Aspergillus activity.

TABLE 2

| | | Anti-Aspergillus |
|---|---|---|
| | Anti-Candida | activity |
| Test compound | (μg/mL) | (μg/mL) |
| Parent compound | 0.20 | 0.39 |
| (compound of Reference Example 1) | | |

### INDUSTRIAL APPLICABILITY

According to the present invention, the compound according to the present invention represented by formula I serves as a prodrug of a parent compound that is an active form, and 1) acts against the onset, development and persistence of infections by inhibiting fungal GPI biosynthesis, thereby inhibiting expression of cell wall proteins and blocking cell wall assembly while preventing the fungus from attaching to cells so that the pathogen cannot become pathogenic, and 2) is also superior in terms of physical properties, and particularly its solubility in water and stability in an aqueous solution, and its in vivo pharmacokinetics and safety, which makes this compound extremely useful in the prevention and treatment of fungal infections.

1. A compound represented by the following formula (I), or a salt thereof: 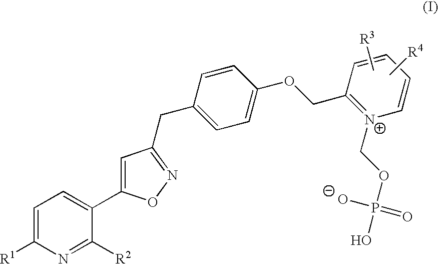
wherein R¹ represents a hydrogen atom, a halogen atom, an amino group, a C₁₋₆ alkyl group, a C₁₋₆ alkoxy group, or a C₁₋₆ alkoxy C₁₋₆ alkyl group;
R² represents a hydrogen atom, a C₁₋₆ alkyl group, an amino group, or a di-C₁₋₆ alkylamino group;
R³ represents a hydrogen atom, a halogen atom, or a C₁₋₆ alkyl group; and
R⁴ represents a hydrogen atom, a halogen atom, or a C₁₋₆ alkyl group.

2. The compound or salt thereof according to claim 1, wherein R² represents an amino group.

3. The compound or salt thereof according to claim 1 or 2, wherein R¹ represents a hydrogen atom.

4. The compound or salt thereof according to claim 1 or 2, wherein R¹ represents an amino group.

5. The compound or salt thereof according to claim 1, wherein R³ represents a hydrogen atom, R⁴ represents a hydrogen atom, a halogen atom, or a C₁₋₆ alkyl group.

6. A 2-((4-((5-(2-amino-3-pyridinyl)-3-isoxazolyl)methyl)phenoxy)methyl)-1-((phosphonooxy)methyl)pyridinium compound represented by the following formula, or a salt thereof:

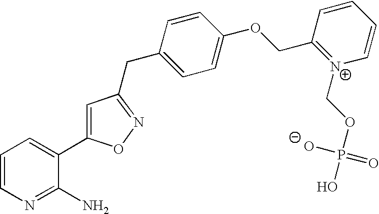

7. A pharmaceutical composition comprising the compound according to claim 1, or a salt thereof.

8. A medicament comprising the compound according to claim 1, or a salt thereof.

9. An antifungal agent comprising the compound according to claim 1, or a salt thereof, as an active ingredient.

10. A method for preventing and/or treating a fungal infection comprising administering a pharmacologically effective amount of the compound according to claim 1, or a salt thereof.

11. Use of the compound according to claim 1, or a salt thereof, for manufacturing an antifungal agent.
